(19) United States
(12) Patent Application Publication
(10) Pub. No.: US 2005/0021093 A1
(43) Pub. Date: Jan. 27, 2005

(54) Subcutaneous lead system for detection and treatment of malignant ventricular arrhythmia

(75) Inventor: Ward M. Brown, La Crosse, WI (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: Team Brown, LLC, La Crosse, WI (US)

(21) Appl. No.: 10/870,278

(22) Filed: Jun. 17, 2004

Related U.S. Application Data
(60) Provisional application No. 60/479,196, filed on Jun. 17, 2003.

(51) Int. Cl. A61N 1/36

(52) U.S. Cl. 607/4

(57) ABSTRACT

A method and apparatus for treating malignant ventricular arrhythmias is provided. The apparatus includes a lead system that is placed subcutaneously in a patient and a pulse generator connected to lead system by lead. The lead system includes a plurality of energy delivery electrodes for delivering energy stimulation to a patient's heart and a plurality of monitoring electrodes for monitoring the occurrence of or sensing an arrhythmia. The lead system is encapsulated in a sheath of biocompatible material to prevent electrodes from contacting each other and shorting out or shorting together.

### BACKGROUND OF THE INVENTION

This invention relates to a method and apparatus for treating malignant ventricular arrhythmias. More particularly, the present invention relates to a subcutaneous lead system for use in the delivery of acute tachyarrhythmia and bradyarrhythmia therapy.

### BRIEF SUMMARY OF THE INVENTION

The use of implantable systems to treat patients that are at risk for life-threatening arrhythmias is well known. Rapid heart rhythms are commonly referred to as tachyarrhythmias. Tachyarrhythmias are defined as any disturbance of the heart's rhythm, regular or irregular, resulting in a rate of over 100 beats per minute. Malignant tachyarrhythmias are many times (generally) treated using implantable defibrillators, the use of which are well known in the art. These systems detect the presence of tachyarrhythmia conditions by monitoring the electrical and mechanical heart activity (such as intra-myocardial pressure, blood pressure, impedance, stroke volume or heart movement) and/or the rate of the electrocardiogram. Defibrillators typically require that one or more defibrillation electrodes be positioned within or on the atrium and/or ventricle of a patient's heart using current endocardial or epicardial lead placement techniques. The use of such defibrillator systems provides consistent long-term monitoring capabilities, and relatively good protection against life-threatening tachyarrhythmias.

Slow heart rhythms are commonly referred to as bradyarrhythmias. Bradyarrhythmias are defined as any disturbance of the heart's rhythms resulting in a rate under 60 beats per minute and are may be (generally) treated using implantable pulse generators. As with devices that treat tachyarrhythmias, most implantable pulse generators that treat bradyarrhythmias generally require leads that are implanted within or on one or more cardiac chambers.

Although the use of endocardial leads placed within the cardiac chambers of a patient's heart provides the capability to deliver a long-term arrhythmia therapy, there are disadvantages associated with such treatments. The placement of these leads requires a relatively time-consuming, costly procedure that is not without risks to the patient including among others: infection, the possibility of vascular perforation, the possibility of perforation and collapse of a lung, and tamponade. In addition, not all patients present for the placement of leads within the cardiac chamber. For example, patients with artificial mechanical tricuspid valves are generally not candidates for leads because of the potential of interference with the proper mechanical functioning of the valves. Similarly, patients with occluded venous access and patients with congenital heart defects do not adapt well to the placement of leads within the cardiac chamber.

One alternative to endocardial and epicardial leads involves subcutaneously-placed electrode systems. The successful treatment and defibrillation of malignant ventricular arrhythmias is dependent on a multitude of different factors, including time to detection and treatment, energy delivery, and “patch” size and placement. In particular, “patch” size and placement are critical to the successful treatment of arrhythmias. More particularly, successful defibrillation relates directly to energy delivery and the muscle mass receiving this energy. By maximizing the muscle mass between the various electrodes defibrillation can be better controlled and delivered. In order to allow more effective sensing, an increased number of sensing electrodes could be used, thus generating multiple data points.

What is needed, therefore, is a new and useful lead system and method of treatment that can provide for various types of arrhythmias, provide appropriate and successful defibrillation and sensing, maximize the muscle mass which it contacts, and also overcome the problems associated with cardiac placement of endocardial and epicardial leads.

The current invention provides a system and method for the long-term monitoring and acute treatment of arrhythmias utilizing a novel lead system placed subcutaneously. The lead system includes a plurality of energy delivery electrodes for delivering energy stimulation to the patient's heart and a plurality of monitoring electrodes for monitoring the occurrence of or sensing an arrhythmia. In operation, the lead system is coupled to an implantable pulse generator or defibrillator for providing electrical stimulation to a patient. The stimulation may include cardioversion or defibrillation shocks and/or pacing pulses to the energy delivery electrodes. The electrical stimulation may be provided between multiple electrodes, or between one or more electrodes. The plurality of electrodes may be housed in a sheath of biocompatible material and may comprise a removable sleeve or a coating that can be peeled or scraped to expose varying amounts of electrode surface area depending on the size of the patient and/or the desired amount of muscle mass to be contacted. The lead system is significantly larger than currently commercially available subcutaneously electrodes and may be implanted proximate subcutaneous tissue at different locations in the patient's body. The increased size of the lead system also allows for multiple sensing electrodes to be utilized, thus allowing for more accurate sensing of rhythms. Lastly, the lead system is flexible to permit the easy positioning in the subcutaneous tissue but rigid enough to maintain its integrity.

According to another embodiment of the invention, a method of therapy is provided. This method includes monitoring the patient's cardiac signals for a condition such as an arrhythmia, and thereafter delivering an electrical therapy to a patient via a subcutaneous electrode system if the condition is detected. Other aspects of the invention will become apparent from the drawings and the accompanying description.

### BRIEF DESCRIPTION OF THE DRAWINGS

FIG. 1 illustrates an exemplary subcutaneous lead system and pulse generator in accordance with the present invention.

FIG. 2 is a top view of a lead system depicting a plurality of energy delivery and monitoring or sensing electrodes housed in a sheath in accordance with one aspect of the present invention.

FIG. 3 is a block diagram illustrating a lead system in accordance with the present invention positioned around a patient's side, with the system extending to the patient's back.

FIG. 4 is a block diagram illustrating a lead system in accordance with the present invention positioned on patients back in a more superior position.

FIG. 5 is a block diagram illustrating a lead system in accordance with the present invention positioned around a patient's side and extending to the patient's back in a more inferior position on the patient's lower back.

### DETAILED DESCRIPTION OF THE INVENTION

The present invention provides a system and method for the long-term monitoring of arrhythmias utilizing a lead or leads including a plurality of energy delivery and sensing electrodes. The invention also provides acute therapy delivery in the event an arrhythmia episode is detected. According to one embodiment of the invention, an implantable pulse generator is also provided. The pulse generator is coupled to at least one subcutaneously-placed lead system in accordance with the present invention. Cardioversion/defibrillation pulses and/or pacing pulses may be delivered between the lead system and the pulse generator/defibrillator, or between two subcutaneously-placed lead systems.

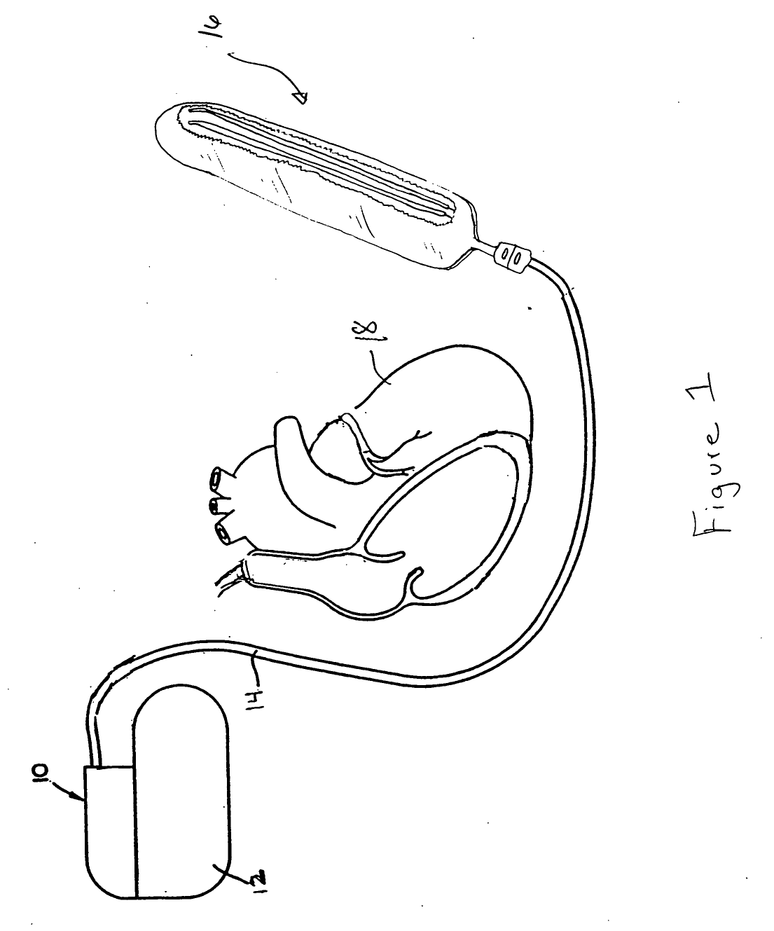

Referring to FIG. 1, an implantable pulse generator 10 and an exemplary lead system 16 in accordance with the present invention is illustrated. Pulse generator 10 includes a device housing 12, and is further coupled to a lead 14 which may be implanted subcutaneously in the left chest or on the back as discussed below. A subcutaneous lead system 16, in accordance with the present invention is operably connected to lead 14. This type of subcutaneous lead system may be positioned subcutaneously, proximal the left ventricular cavity on the patient's chest, on the patient's side or back, or any other portion of the body appropriate for providing electrical stimulation to the heart. In operation, electrical stimulation in the form of cardioversion/ defibrillation may be delivered to heart 18 between device 10 and lead system 16. Alternatively, pacing pulses may be delivered between the pulse generator/defibrillator 10 and lead system 16.

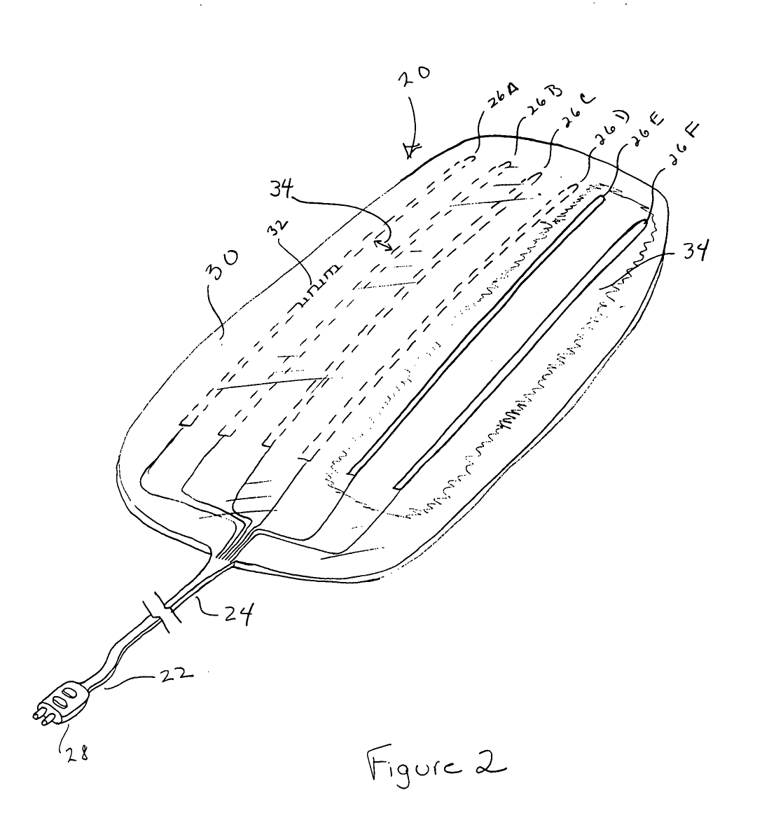

Referring to FIG. 2, a top view of a lead system 20 in accordance with the present invention is shown. Lead system 20 is coupled to distal end 22 of connecting wire 24. In one embodiment of the present invention, lead system 20 includes a plurality of electrodes 26A-26F. Electrodes 26A-26F are comprised of energy delivery electrodes that deliver energy and sensing or monitoring electrodes that sense the onset of arrhythmia. More or fewer of these electrodes may be provided depending upon whether the lead system 20 is an adult or pediatric system or the type of treatment and monitoring the prescribing physician desires. In addition, the two different types of electrodes, i.e. energy delivery and sensing, may be placed alternately in the device in an energy delivery, sensing, energy delivery, sensing, etc. configuration or in other configurations. For example, other embodiments may include placing the electrodes in a sensing, sensing, energy delivery, energy delivery configuration. The number of configurations of electrodes 26A-26F within lead system 20 are virtually limitless and are intended to be encompassed within the scope of the present invention.

In yet another embodiment of the present invention, each energy delivery electrode may include a defibrillation coil 32 wrapped circumferentially around the electrode 26A. When connector 28 is coupled via lead 14/24 to a pulse generator, a cardioversion/defibrillation pulse may be provided via one or more of the electrodes 26A-26F. In one embodiment, the electrodes that are activated may be selected via a switch provided by the lead 14/24.

Lead system 20 may include one or more sensing electrodes 26A-26F for sensing cardiac signals. This electrode may be used in a unipolar mode wherein signals are sensed between one electrode and the device 10. Alternatively, sensing may be performed between the sensing electrode and one of the energy delivery electrodes or other sensing electrodes present in the lead system 20. Preferably, it is desirable to include a plurality of sensing electrodes in the lead system of the present invention so that multiple sensing data points are obtained. Multiple data points enable more accurate detection of abnormal and life-threatening cardiac rhythms. In addition, the use of a plurality of sensing electrodes in the lead system and multiple data point output allows for a more accurate differentiation between abnormal cardiac rhythms and normal muscle movement. As would be expected, having only one sensing electrode limits the ability to differentiate the various signals that may be detected.

Lead system 20 and each electrode 26A-26F may be encapsulated in a sheath 30 of biocompatible material such as urethane, polycarbonate, acetyl, nylon, PTFE/teflon, polyimides, polyamides, polyethylene, polysulfone, polypropylenes and mixtures thereof. Sheath 30 is provided to prevent electrodes 26A-26F from coming in contact with each other and possibly shorting out and/or shorting together. Sheath 30 may be scratched or peeled off 34 exposing more of the surface of any of the electrodes 26A-26F depending on patient need and prescribing physician desires. For example, it may be desirable to expose more of any of the electrodes 26A-26F to a greater muscle mass. This is easily accomplished by having a sheath 30 that is readily removable in whole or in part by peeling or scratching the surface. In another embodiment, sheath 30 may comprise a sleeve that is removable wholly or partially prior to subcutaneously placement in the patient. In this alternative embodiment, biocompatible spacers may be provided between electrodes 26A-26F to ensure that they do not short out or short together. Alternatively, the diameter of the space 34 between each electrode may be of sufficient width to prevent shorting out of adjacent electrodes. In this case, an appropriate range of widths are from 2.0 mm to 90.0 mm.

In one embodiment of the present invention, electrodes 26A-26F of lead system 20 are made of flexible materials to allow for malleable insertion subcutaneously and ideal placement on muscle mass. Representative examples of suitable materials include, nickel, titanium, stainless steel, certain grades of nitinol and mixtures thereof. It is preferred that electrodes 26A-26F are made from conductive materials. In an alternative embodiment only a portion of the electrode may be made from conductive materials, such as a conductive coil circumferentially surrounding the electrode or a “cap” or “head” portion of the electrode may be made of conductive materials while the “body” of the electrode may comprise non-conductive material. In use, lead system 20 is positioned under the skin on a patient's chest, side, back, or any other point of the body as required. Insulative spacers may be located between the electrodes 26A-26F, if desired, to prevent them from shorting together. In an alternative embodiment, a sheath 30 of biocompatible material may be utilized. If desired, multiple such lead systems 20 may be used in conjunction with the present invention. For example, one lead system 20 may be positioned on the chest over the left ventricle, while another lead system is positioned behind the left ventricle on the back. Cardioversion/defibrillation shocks or pacing pulses may be delivered between the two lead systems 20. Alternatively, electrical stimulation may be provided between one or more lead systems 20 and the device housing 12.

The overall shape and design of the lead system 20 of the present invention is important to the delivery of successful defibrillation. Successful defibrillation involves the ability to deliver appropriate signals that will create non-threatening heart rhythms and relates directly to the amount of energy delivered and the muscle mass receiving the energy. In order to provide efficient defibrillation signals, the muscle mass encompassed by or in contact with the various electrodes 26A-26F comprising lead system 20 must be of sufficient size to cause effective delivery of energy. As presently contemplated, lead system 20 would be approximately 5-8 cm in diameter but may be larger or smaller depending on the actual size of the patient. In addition, it is contemplated that pediatric versions of the lead system 20 in accordance with the present invention would be on a scale smaller than the adult version but still be comparatively larger than lead system currently commercially available.

Electrodes 26A-26F used with the present invention 20 may be any of the electrode types now known or known in the future for subcutaneous delivery of electrical stimulation. Such electrodes may be coated with biologically-active agents such as glucocorticoids (e.g. dexamethasone, beclamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents. Such coating may be useful to prevent excessive tissue in-growth. Such electrodes may further include a low-polarization coating such as TiN. Alternatively, the electrodes may be coated with an antibiotic or other biologically-active agent used to prevent infections and inflammation.

As described above, in one embodiment of the present invention a pulse generator is coupled to one or more subcutaneous lead systems having a plurality of energy delivery electrodes and a plurality of sensing electrodes. The electrodes provide electrical stimulation to a patient based on sensed cardiac signals. The sensed signals may be obtained using a selected pair of sensing electrodes, which may reside on one or more of the leads coupled to pulse generator 10, or on the device housing 12 itself.

Although all of the foregoing examples illustrate a lead system 20 including six electrodes, it is anticipated that fewer than or more than six electrodes may be provided. In one embodiment, seven or more electrodes may be coupled or adjacent to the device, while in another embodiment five or four electrodes may be utilized. In each case, the physician may select which of the electrodes will be activated for a given patient. In one embodiment, cardiac signals are sensed between a selected pair of the electrodes based on a signal optimization method. Regardless of which one or more electrodes or electrode pairs are selected for monitoring purposes, the sensed cardiac signals may be analyzed to detect the presence of an arrhythmia. If an arrhythmia is detected, appropriate therapy may be administered. As described above, the lead system in accordance with the present invention includes defibrillation electrodes. If multiple data points collected from the sensing or monitoring electrodes indicate the presence of a tachyarrhythmia or ventricular fibrillation, a high-voltage shock may be delivered between one or more of the subcutaneous defibrillation electrodes. The monitoring electrodes would then determine whether the arrhythmia or fibrillation has terminated. If not, another shock may be delivered. This therapy will continue until normal rhythm has been restored.

As described above, therapy for bradyarrhythmia may be provided in addition to, or instead of, the tachyarrhythmia therapy. In this embodiment, lower-voltage pulses for pacing therapy for bradyarrhythmias are delivered. These lower-voltage pulses could be on the order of between 50 and 150 volts, for example. In one embodiment, these pulses have amplitude of around 100 volts. Monitoring for a bradyarrhythmia could be accomplished using the sensing electrodes discussed above. For example, the device may be programmed to detect a period of a systole that is greater than a predetermined period, such as three seconds. When a period greater than this length is detected, the output circuit of the device 10 is charged to the pacing voltage. A pacing pulse may then be delivered to the energy delivery electrodes. The sensing electrodes monitor the cardiac waveform to ensure that the pacing pulse is only delivered during predetermined periods of the cardiac cycle. For example, delivery of the pulse should not occur during the occurrence of a T-wave.

Following delivery of a pacing pulse, the output circuit begins charging in preparation for delivery of another pulse while monitoring of the cardiac signals continues via the plurality of sensing electrodes.

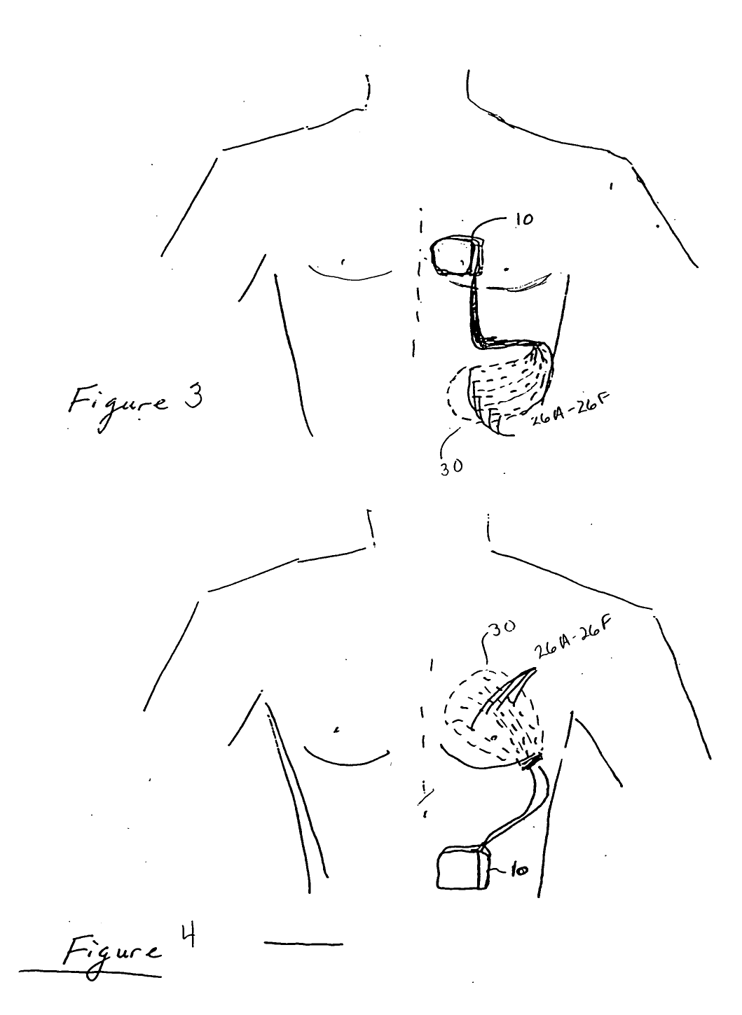

FIGS. 3 through 5 illustrate various exemplary modalities of utilizing the present invention.

FIG. 3 is a block diagram illustrating a lead system 20 positioned around a patient's side, with electrodes 26A-26F extending to and placed on the patient's back. Electrical stimulation is delivered between the device can 10, which is positioned over the left ventricle, and the electrodes.

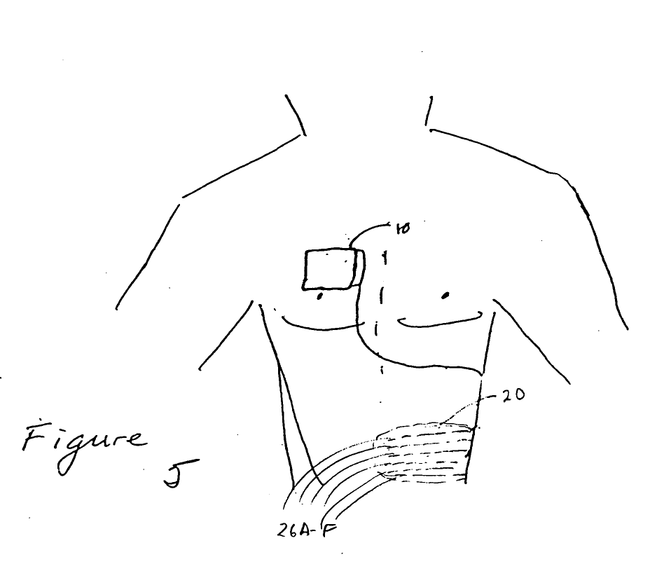

FIG. 4 is a block diagram illustrating a lead system 20 in accordance with the present invention positioned on a patient's back in a more superior position than is shown in FIG. 3. Electrical stimulation may be delivered between the device can 10, which is positioned in the abdominal cavity, and the electrodes 26A-26F of the lead system 20.

FIG. 5 is a block diagram illustrating a lead system 20 positioned around a patient's side, with electrodes 26A-26F extending to the patient's back in a more inferior position than is shown in FIG. 3 or 4. Electrical stimulation is delivered between the device can 10, which is positioned proximal the right side of the heart and the electrodes 26A-26F.

The above-described inventive system and method provides a therapy that avoids the risks of transvenous lead delivery. Such a system may be used for patients that are at-risk for arrhythmias, but have not yet experienced a confirmed arrhythmic episode. The device may therefore provide a needed long-term monitoring function, as well as any interventional therapy that is required.

The present invention provides an improved lead system and method of treatment that enables the treatment and monitoring of various types of arrhythmias, provides appropriate and successful defibrillation and sensing, and maximizes the muscle mass that it contacts. As discussed above, the inventive system provides many important benefits over other conventional systems for some patients. The procedure is faster because there is no need for venous or epicardial access, and therefore the procedure is less invasive, and would not require procedures needing sophisticated surgical facilities and devices. Additionally, the implant procedure can be accomplished without exposing the patient to potentially-harmful radiation that accompanies fluoroscopy. The risk of infection is reduced, and the procedure may be provided to patients that are contraindicated for a more traditional device. Additionally, the system is more comfortable than externally-worn devices. The system is well suited for both adult and pediatric use.

The foregoing detailed description of the preferred embodiments of the invention has been provided for the purposes of illustration and description. It is not intended to be exhaustive or to limit the invention to the precise embodiments disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

1. A system for providing arrhythmia therapy to a patient, comprising:
(i) an implantable pulse generator; and
(ii) a lead system operably coupled to the implantable pulse generator, said lead system including at least one energy delivery electrode and at least one monitoring electrode, wherein said at least one energy delivery electrode delivers electrical stimulation to the patient upon detection by said at least one monitoring electrode of an arrhythmia.

2. The system for providing arrhythmia therapy to a patient as recited in claim 1, wherein said lead system includes a biocompatible sheath housing said at least one energy delivery electrode and said at least one monitoring electrode.

3. The system for providing arrhythmia therapy to a patient as recited in claim 2, wherein said sheath is removable.

4. The system for providing arrhythmia therapy to a patient as recited in claim 2, wherein said biocompatible sheath comprises a material selected from the group consisting essentially of urethane, polycarbonate, acetyl, nylon, polytetrafluoroethylene, polyimides, polyamides, polyethylene, polysulfones, polypropylenes, and mixtures thereof.

5. The system for providing arrhythmia therapy to a patient as recited in claim 2 wherein said sheath partially exposes said plurality of energy delivery electrodes and said plurality of monitoring electrodes.

6. The system for providing arrhythmia therapy to a patient as recited in claim 1 further comprising a plurality of energy delivery electrodes.

7. The system for providing arrhythmia therapy to a patient as recited in claim 1 further comprising a plurality of monitoring electrodes.

8. The system for providing arrhythmia therapy to a patient as recited in claim 6 further comprising biocompatible insulative spacing means, said spacing means positioned between said plurality of energy delivery electrodes for preventing said electrodes from shorting.

9. The system for providing arrhythmia therapy to a patient as recited in claim 1 further comprising a plurality of energy delivery electrodes and a plurality of monitoring electrodes.

10. The system for providing arrhythmia therapy to a patient as recited in claim 1 wherein said at least one energy delivery electrode and said at least one monitoring electrode are flexible.

11. The system for providing arrhythmia therapy to a patient as recited in claim 9 wherein said plurality of energy delivery electrodes and plurality of monitoring electrodes are coated with a biologically-active agent selected from the group consisting essentially of glucocorticoids, heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, anti-inflammatory agents and combinations thereof.

12. The system for providing arrhythmia therapy to a patient as recited in claim 6 further comprising a defibrillation coil circumferentially positioned about each of said plurality of energy electrodes.

13. A system for providing arrhythmia therapy to a patient, comprising:
(i) an implantable pulse generator; and
(ii) a lead system operably coupled to the implantable pulse generator, said lead system including at least one energy delivery electrode and a plurality of monitoring electrode means for monitoring arrhythmia, wherein said at least one energy delivery electrode delivers electrical stimulation to the patient upon detection by said plurality of monitoring electrode means of an arrhythmia.

14. The system for providing arrhythmia therapy to a patient as recited in claim 12 wherein said lead system operably coupled to the implantable pulse generator includes a plurality of lead systems.

15. A method for treating patient arrhythmias comprising:
(i) providing an implantable pulse generator;
(ii) providing a lead system operably coupled to the implantable pulse generator, said lead system including a plurality of energy delivery electrodes and a plurality of monitoring electrodes, said plurality of energy delivery electrodes to deliver electrical stimulation to the patient upon detection by said plurality of monitoring electrodes of an arrhythmia.

16. The method for treating patient arrhythmias as recited in claim 15 further comprising providing biocompatible sleeve housing means for housing said plurality of energy delivery electrodes and said plurality of monitoring electrodes.

17. The method for treating patient arrhythmias as recited in claim 16 wherein said biocompatible sleeve housing means is removably received on said plurality of energy delivery electrodes and said plurality of monitoring electrodes.

18. The method for treating patient arrhythmias as recited in claim 16 wherein said biocompatible sleeve housing means partially exposes said plurality of energy delivery electrodes and said plurality of monitoring electrodes.

19. The method for treating patient arrhythmias as recited in claim 16 further comprising providing a defibrillation coil circumferentially positioned about each of said plurality of energy electrodes.

20. The method for treating patient arrhythmias as recited in claim 16 further comprising providing biologically active coating means for coating said plurality of energy delivery electrodes and said plurality of monitoring electrodes.
